(19) United States
(12) Patent Application Publication
(10) Pub. No.: US 2026/0158123 A1
(43) Pub. Date: Jun. 11, 2026

(54) METHODS AND COMPOSITIONS FOR TREATING FRUCTOSE INTOLERANCE AND FRUCTOSE MALABSORPTION

(71) Applicant: Anagram Therapeutics, Inc., Framingham, MA (US)

(72) Inventors: Alexey Margolin, Newton, MA (US); Bhami Shenoy, South Grafton, MA (US)

(21) Appl. No.: 18/683,426

(22) PCT Filed: Aug. 12, 2022
(86) PCT No.: PCT/US2022/074934

§ 371 Date: Feb. 13, 2024

Related U.S. Application Data
(60) Provisional application No. 63/232,922, filed on Aug. 13, 2021.

(51) Int. Cl. A61K 38/52; A61K 38/44

(52) U.S. Cl. CPC A61K 38/52; A61K 38/44; A61K 38/443; C12Y 101/03004; C12Y 111/01006; C12Y 503/01005

(57) ABSTRACT

Disclosed are pharmaceutical compositions comprising a glucose(xylose) isomerase, glucose oxidase enzyme and a peroxide-degrading enzyme (e.g., a catalase enzyme), which can be used, among other things, to treat fructose intolerance and fructose malabsorption.

### CROSS-REFERENCE TO RELATED APPLICATIONS

This application claims the benefit of and priority to U.S. Provisional Patent Application No. 63/232,922, filed on Aug. 13, 2021, the disclosure of which is hereby incorporated by reference in its entirety for all purposes.

### FIELD OF THE INVENTION

The invention relates generally to methods and compositions for treating fructose intolerance and fructose malabsorption, and, more particularly, the invention relates to compositions containing a glucose(xylose) isomerase enzyme, glucose oxidase enzyme, and a peroxide-degrading enzyme (e.g., a catalase enzyme), and their use in treating fructose intolerance and fructose malabsorption.

### BACKGROUND

Increased sugar consumption is considered to be a contributor to obesity and diabetes and their associated cardiometabolic risks. As a result of its unique metabolic properties, the fructose component of sugar may be particularly harmful. Diets high in fructose can create all of the key features of metabolic syndrome.

Fructose is a six-carbon monosaccharide (a polyhydroxyketone) that is widely consumed in the Western diet. Fructose is typically ingested in three forms: as the pure monosaccharide; as the disaccharide sucrose, including glucose bonded to fructose, which can be hydrolyzed by the enzyme sucrase; and as polymerized forms such as oligosaccharides and polysaccharides.

Polymerized forms of fructose are variably described as inulins, fructans, and fructo-oligosaccharides. Fructose in its various forms is believed to be associated with irritable bowel syndrome (IBS), a contributor to the obesity crisis (particularly in the U.S.), and involved in the pathogenesis of nonalcoholic fatty liver disease. Lambertz et al. (2017) FRONTIERS IN IMMUNOLOGY 8: 1, DiNicolantonio and Lucan (2015) MED. HYPOTHESES 85(3): 295.

There has been a marked rise in fructose consumption in the U.S. This high level of fructose consumption is believed to result from the use of high fructose corn syrups (HFCS) as a sweetener in food manufacturing. HFCS is a cheaper alternative to sucrose and includes up to 80% fructose. Gibson et al. (2007) ALIMENT. PHARMACOL THER. 25: 349. Sugar-sweetened beverages (SSBs) are a major source of added sugar in diets worldwide and include sodas, fruit-flavored drinks, and sport drinks. On average, SSBs contribute approximately 7% of daily calories and nearly 50% of added sugars in the diet. Hannou et al. (2018) J. CLIN. INVEST. 128(2): 545, Tappy and Le (2010) PHYSIOL. REV. 90: 23.

There are two distinct types of fructose intolerance—Hereditary Fructose Intolerance (HFI) and Dietary Fructose Intolerance (DFI). Hou et al. (2019) FRONTIERS IN GENETICS 10: 1. HFI is a rare genetic disorder of fructose metabolism that is related to a deficiency of the enzyme aldolase B (ALDOB) and intracellular accumulation of toxic fructose 1-phosphate in the liver, intestine, and kidney. Ingestion of fructose causes acute symptoms such as vomiting and diarrhea, but also life-threatening manifestations, including acute hypoglycemia, lethargy, renal tubularacidosis, and acute liver failure. Di Dato et al. (2019) NUTRIENTS 11: 2397. Chronic ingestion of fructose results in poor feeding, failure to thrive or growth retardation, liver and kidney damage. If diagnosed and treated early, HFI is a preventable, however, without prompt intervention, it can be lethal. DFI includes situations in which free fructose is available for fermentative metabolism by luminal bacteria before it can be absorbed across the small intestinal mucosa.

The vast majority of fructose absorption occurs via two major transporters known as GLUT5 and GLUT2. Ferraris et al. (2018) ANNU. REV. NUTR. 38: 41. GLUT5 is a facultative transporter (it depends upon a concentration gradient for movement of substrate across the transporter) and is specific for fructose. It is found in the apical membrane. As fructose is cleared rapidly from the circulation, luminal uptake of fructose is ensured. Although this uptake mechanism has low capacity, the transporters are present along the length of the small intestine. GLUT2 is a low affinity, facultative transporter that can transport glucose, fructose and galactose. It is present constitutively on the basolateral membrane, where it transports hexoses down a concentration gradient out of the cell. The intestine's capacity to absorb fructose is saturable, and a healthy adult's ability to absorb free fructose typically ranges from less than 5 g to more than 50 g per day. Ferraris et al. (2018) ANNU. REV. NUTR. 38: 41. Unabsorbed fructose can impose an osmotic load on the distal small intestine and the colon, which may contribute to gastrointestinal symptoms. Moreover, fructose can serve as a substrate for bacterial fermentation, leading to formation of gas and other bacterial metabolites, which can affect intestinal motility and cause various symptoms such as abdominal pain and bloating. Excessive fructose consumption may have significant effects on lipid metabolism, contributing both to steatosis and to increased circulating triglyceride levels in the form of very low-density lipoprotein, induction of hyperinsulinemia, increased adiposity and hypertension.

To date, the only available therapy for fructose intolerance and fructose malabsorption is a fructose, sorbitol, and sucrose (FSS)-free diet. Total exclusion of these sugars is not easily feasible because of small hidden fructose amounts contained in many foods. Accordingly, there is an ongoing need for new and effective pharmaceutical compositions and therapies for regulating fructose absorption and for treating fructose intolerance (e.g., Hereditary Fructose Intolerance and Dietary Fructose Intolerance) and fructose malabsorption.

### SUMMARY OF THE INVENTION

The invention is based, in part, of the discovery of pharmaceutical compositions comprising a glucose(xylose) isomerase (also known as fructose isomerase) enzyme, a glucose oxidase enzyme, and a peroxide-degrading enzyme (e.g., a catalase enzyme) that can be used to treat fructose intolerance (e.g., Hereditary Fructose Intolerance or Dietary Fructose Intolerance), and/or fructose malabsorption in a subject in need thereof.

Accordingly, in one aspect, the invention provides a pharmaceutical composition comprising (or consisting essentially of) a glucose(xylose) isomerase enzyme, a glucose oxidase enzyme, a peroxide-degrading enzyme (e.g., a catalase enzyme), and a pharmaceutically acceptable excipient.

In certain embodiments, the glucose(xylose) isomerase enzyme is spray-dried or lyophilized. The glucose(xylose) isomerase enzyme can be a microbial glucose(xylose) isomerase enzyme, or a functional fragment or variant thereof. The glucose(xylose) isomerase enzyme can be derived from Streptomyces murinus. In certain embodiments, the glucose(xylose) isomerase enzyme comprises SEQ ID NO: 5, or a functional fragment or variant thereof. In certain embodiments, the pharmaceutical composition comprises from about 1,000 to about 250,000 international units (I.U.) of the glucose(xylose) isomerase enzyme.

In certain embodiments, the glucose oxidase enzyme is spray-dried or lyophilized. The glucose oxidase enzyme can be a microbial glucose oxidase enzyme, or a functional fragment or variant thereof. The glucose oxidase enzyme can derived from Aspergillus niger, for example, the glucose oxidase enzyme comprises SEQ ID NO: 1, SEQ ID NO: 2, or a functional fragment or variant thereof. In certain embodiments, the pharmaceutical comprises from about 1,000 to about 250,000 international units (I.U.) of the glucose oxidase enzyme.

In certain embodiments, the peroxide-degrading enzyme is spray-dried or lyophilized. In certain embodiments, the peroxide-degrading enzyme can be a catalase enzyme or a peroxidase enzyme. In certain embodiments, the peroxide-degrading enzyme is a catalase enzyme, which can be microbial catalase enzyme, or a functional fragment or variant thereof. In certain embodiments, the catalase enzyme is derived from Aspergillus niger, e.g., the catalase enzyme comprises SEQ ID NO: 3, SEQ ID NO: 4, or a functional fragment or variant thereof. In certain embodiments, the pharmaceutical comprises from about 1,000 to about 250,000 international units (I.U.) of the catalase enzyme.

Depending upon the circumstances, the ratio of international units of the glucose(xylose) isomerase to international units of the glucose oxidase enzyme in the pharmaceutical composition is in the range from 0.1 to 10. In addition or in the alternative, the ratio of international units of glucose oxidase to international units of the peroxide-degrading enzyme in the pharmaceutical composition is in the range from 0.1 to 10.

In certain embodiments, the pharmaceutical composition is formulated as an oral dosage form. The pharmaceutical composition can be formulated as a solid oral dosage form, for example, a powder, satchel, granulate, pellet, micropellet, tablet, or minitablet. Alternatively, the pharmaceutical composition can be formulated as a liquid oral dosage form, for example, an elixir, syrup, or drop. The pharmaceutical composition can have a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

In another aspect, the invention provides a method of treating fructose intolerance (e.g., dietary fructose intolerance or hereditary fructose intolerance) in a subject in need thereof. The method comprises administering (for example, orally administering) to the subject an effective amount of any of the enzymes or compositions described herein, to treat the fructose intolerance in the subject.

In another aspect, the invention provides a method of treating irritable bowel syndrome (IBS), FODMAP (fermentable oligosaccharides, disaccharides, monosaccharides, and polyols) intolerance or malabsorption, obesity, metabolic syndrome, diabetes, hyperglycemia, or hyperinsulinemia in a subject in need thereof. The method comprises administering (for example, orally administering) to the subject an effective amount of any of the enzymes or compositions described herein, to treat the fructose intolerance in the subject.

In another aspect, the invention provides a method reducing a fructose level in a subject in need thereof. The method comprises administering (e.g., orally administering) to the subject an effective amount of any of the enzymes or compositions described herein, to reduce the fructose level in the subject. In certain circumstances, the method reduces the level of fructose in the blood of the subject and/or in the gastrointestinal tract of the subject. Alternatively or in addition, the method reduces the level of fructose in a sample (e.g., a body fluid sample, e.g., blood, serum or plasma) from the subject.

In certain embodiments, the method reduces the level of fructose in the blood of the subject from 0 to 240 minutes or 0 to 360 minutes following a meal or snack, as determined by an Area Under Curve (AUC) analysis, by at least about 10%, 20%, 30%, 40%, 50%, 60%, 70%, 80%, or 90%. In certain embodiments, the method reduces fructose Cmax in the blood of the subject following a meal or snack by at least about 10%, 20%, 30%, 40%, 50%, 60%, 70%, 80%, or 90%. In certain embodiments, the method reduces fructose Tmax in the blood of the subject following a meal or snack by at least about 10%, 20%, 30%, 40%, 50%, 60%, 70%, 80%, or 90%. In certain embodiments, when administered together with a food, the method reduces the caloric intake of the food by at least about 10%, 20%, 30%, 40%, 50%, 60%, 70%, 80%, or 90%.

In certain embodiments of any of the foregoing methods, the subject has dietary fructose intolerance, hereditary fructose intolerance, irritable bowel syndrome (IBS), FODMAP (fermentable oligosaccharides, disaccharides, monosaccharides, and polyols) malabsorption and/or intolerance, obesity, metabolic syndrome, diabetes, hyperglycemia, or hyperinsulinemia.

In certain embodiments of any of the foregoing methods, the enzyme or composition is administered to the subject 1, 2, 3, 4, or more than 4 times per day. In certain embodiments, the enzyme or composition is administered to the subject together with a meal or snack (e.g., at each meal or snack).

These and other aspects and features of the invention are described in the following detailed description and claims.

### DESCRIPTION OF THE DRAWINGS

The invention can be more completely understood with reference to the following drawings.

FIG. 1 depicts the effect of glucose(xylose) isomerase, glucose oxidase, and catalase on conversion of fructose (1%) at pH 7.4 and 37° C.

### DETAILED DESCRIPTION

The invention is based, in part, upon the discovery of pharmaceutical compositions comprising a glucose(xylose) isomerase, a glucose oxidase enzyme, and a peroxide-degrading enzyme (e.g., a catalase enzyme) that can be used to treat a variety of disorders including, for example, fructose intolerance (e.g., dietary fructose intolerance or hereditary fructose intolerance), irritable bowel syndrome (IBS), FODMAP (fermentable oligosaccharides, disaccharides, monosaccharides, and polyols) malabsorption and/or intolerance, obesity, metabolic syndrome, diabetes, hyperglycemia, or hyperinsulinemia in a subject in need thereof.

Various features and aspects of the invention are discussed in detail below.

### I. Enzymes

Among other things, the invention provides pharmaceutical compositions including a glucose(xylose) isomerase, a glucose oxidase enzyme and a peroxide-degrading enzyme that, for example, are useful in treating one or more of the disorders described herein. Given that fructose levels can be directly associated with these disorders, the methods and compositions described herein are designed to reduce fructose levels in a subject. The use of the compositions described herein facilitate the conversion of fructose into glucose via the glucose(xylose) isomerase enzyme. However, given the natural equilibrium between fructose and glucose, the glucose oxidase and peroxidase enzymes remove at least a portion of the glucose produced by the glucose(xylose) isomerase enzyme to shift the equilibrium to prevent the glucose being reconverted into fructose.

As used herein, the term “glucose(xylose) isomerase” refers to any enzyme, or a functional fragment thereof, that is capable of catalyzing at least the following reaction:

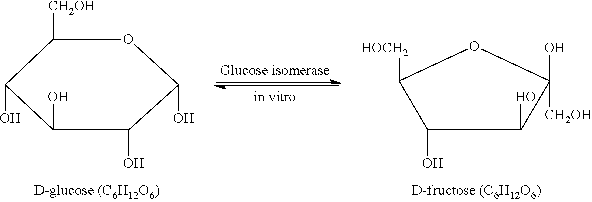

Glucose(xylose) isomerase is also referred to as, for example, D-xylose isomerase, D-xylose ketoisomerase, D-xylose ketol-isomerase, D-xylose ketol isomerase, D-xylose: ketol-isomerase, D-xylulose keto-isomerase, glucose isomerase, glucose/xylose isomerase, EC 5.3.1.5, D-XI, GXI, Maxazyme™, Optisweet™, Sweetase™, and Spezyme™.

The term glucose(xylose) isomerase includes variants having one or more amino acid substitutions, deletions, or insertions relative to a wild-type glucose(xylose) isomerase sequence, and/or fusion proteins or conjugates including a glucose(xylose) isomerase. As used herein, the term “functional fragment” of a glucose(xylose) isomerase refers to fragment of a full-length glucose(xylose) isomerase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring glucose(xylose) isomerase. Glucose(xylose) isomerase enzymatic activity may be assayed by any method known in the art. Exemplary glucose(xylose) isomerase activity include assays are available from Abcam (Cambridge, UK; Glucose Isomerase Activity Assay Kit (Colorimetric); ab273289) and Biovision (Milpitas, CA, USA).

Exemplary glucose(xylose) isomerase enzymes include glucose(xylose) isomerase enzymes derived from Streptomyces murinus. An amino acid sequences of an exemplary wild-type glucose oxidase enzyme derived from Streptomyces murinus is depicted in SEQ ID NO: 5 (UniProtKB—P37031).

Additional exemplary glucose(xylose) isomerase enzymes include glucose(xylose) isomerase enzymes derived from Escherichia coli, Klebsiella pneumoniae, Lactobacillus brevis, Lactobacillus pentosus, Bacillus subtilis, Caldicellulosiruptor bescii, Staphylococcus xylosus, Thermoanaerobacter ethanolicus, Thermoanaerobacter thermosulfurogenes, Arthrobacter sp., Actinoplanes missouriensis, Ampullariella sp., Streptomyces violaceoniger, Streptomyces murinus, Streptomyces rochei, Streptomyces rubiginosus, Streptomyces olivochromogenes, Thermus thermophilus, or Thermotoga neopolitana. Additional exemplary glucose(xylose) isomerase enzymes can be found on the world wide web at brenda-enzymes.org/enzyme.php?ecno=5.3.1.5 #ORGANISM or at brenda-enzymes.org/enzyme.php?ecno=5.3.1.5.

A glucose(xylose) isomerase enzyme may comprise a consensus sequence VX₁WX₂GREGX₃E (SEQ ID NO: 6), wherein X₁ is any amino acid, X₂ is G or P, and X₃ is Y,S,T, A, or E. Alternatively or in addition, a glucose(xylose) isomerase enzyme may comprise a consensus sequence X₁EPKPX₂X₃P (SEQ ID NO: 7), where X₁ is L, I, V, or M, X₂ is any amino acid, and X₃ is E or Q.

As used herein, the term “glucose oxidase” refers to any enzyme, or a functional fragment thereof, that is capable of catalyzing the oxidation of β-D-glucose to D-glucono-ω-lactone and hydrogen peroxide and/or the conversion of D-glucono-ω-lactone to gluconic acid.

Glucose oxidase typically catalyzes at least the following reaction:

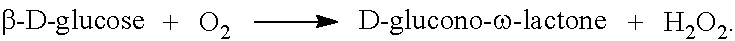

Glucose oxidase is also referred to as EC 1.1.3.4, glucose oxyhydrase, corylophyline, penatin, glucose aerodehydrogenase, microcid, β-D-glucose oxidase, D-glucose oxidase, D-glucose-I-oxidase, β-D-glucose:quinone oxidoreductase, glucose oxyhydrase, and deoxin-1, and, unless indicated otherwise, the terms are used interchangeably herein. The term glucose oxidase includes variants having one or more amino acid substitutions, deletions, or insertions relative to a wild-type glucose oxidase sequence, and/or fusion proteins or conjugates including a glucose oxidase. As used herein, the term “functional fragment” of a glucose oxidase refers to fragment of a full-length glucose oxidase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring glucose oxidase. Glucose oxidase enzymatic activity may be assayed by any method known in the art. Exemplary glucose oxidase activity assays are available from Sigma-Aldrich (Cat. No. MAK097).

Exemplary glucose oxidase enzymes include glucose oxidase enzymes derived from Aspergillus niger. Amino acid sequences of exemplary wild-type glucose oxidase enzymes derived from Aspergillus niger are depicted in SEQ ID NO: 1 (with signal sequence) and SEQ ID NO: 2 (without signal sequence). Additional exemplary glucose oxidase enzymes can be found on the world wide web at brenda-enzymes.org/enzyme.php?ecno=1.1.3.4 #ORGANISM or brenda-enzymes.org/enzyme.php?ecno=1.1.3.4.

As used herein, the term “peroxide-degrading enzyme” refers to any enzyme, or a functional fragment thereof, that is capable of degrading hydrogen peroxide. Exemplary peroxide-degrading enzymes include catalase and peroxidase enzymes.

As used herein, the term “catalase” refers to any enzyme, or a functional fragment thereof, that is capable of catalyzing the decomposition of hydrogen peroxide to water and oxygen. Catalase typically catalyzes the following reaction:

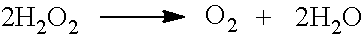

Catalase is also referred to as EC 1.11.1.6, equilase, caperase, optidase, catalase-peroxidase, and CAT, and, unless indicated otherwise, the terms are used interchangeably herein. The term catalase includes variants having one or more amino acid substitutions, deletions, or insertions relative to a wild-type catalase sequence, and/or fusion proteins or conjugates including a catalase. As used herein, the term “functional fragment” of a catalase refers to fragment of a full-length catalase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring catalase. Exemplary catalase activity assays are available from Sigma-Aldrich (Cat. Nos. CAT100 and 219265).

Exemplary catalase enzymes include catalase enzymes derived from Aspergillus niger. Amino acid sequences of exemplary wild-type catalase enzymes derived from Aspergillus niger are depicted in SEQ ID NO: 3 (with signal sequence) and SEQ ID NO: 4 (without signal sequence). Additional exemplary catalase enzymes can be found on the world wide web at brenda-enzymes.org/enzyme.php?ecno=1.11.1.6 #ORGANISM or brenda-enzymes.org/enzyme.php?ecno=1.11.1.6.

As used herein, the term “peroxidase” refers to any enzyme, or a functional fragment thereof, that is capable of catalyzing an oxidation-reduction reaction by the mechanism of a free radical that can transform a compound into an oxidized or polymerized product. Peroxidases typically catalyze the following reaction: H₂O₂+AH₂→2 H₂O+A

The term peroxidase includes variants having one or more amino acid substitutions, deletions, or insertions relative to a wild-type peroxidase sequence, and/or fusion proteins or conjugates including a peroxidase. As used herein, the term “functional fragment” of a peroxidase refers to fragment of a full-length peroxidase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring peroxidase. Exemplary peroxidase activity assays are available from Sigma-Aldrich (Cat. No. MAK092).

Peroxidases represent a large group of enzymes. Exemplary peroxidase enzymes can be found on the world wide web at brenda-enzymes.org/enzyme.php?ecno=1.11.1.7 #ORGANISM or brenda-enzymes.org/enzyme.php?ecno=1.11.1.7.

In certain embodiments, the glucose(xylose) isomerase, glucose oxidase, and/or peroxide-degrading enzyme comprise at least one (for example, one, two, three, four, five, six, seven, or eight) mutation(s) relative to a wild type glucose(xylose) isomerase, glucose oxidase, and/or peroxide-degrading enzyme disclosed herein.

In certain embodiments, the glucose(xylose) isomerase, glucose oxidase, and/or peroxide-degrading enzyme comprise one or more conservative substitutions relative to a glucose(xylose) isomerase, glucose oxidase, and/or peroxide-degrading enzyme disclosed herein. In other embodiments, the glucose(xylose) isomerase, glucose oxidase, and/or peroxide-degrading enzyme comprises one or more non-conservative substitutions relative to a glucose(xylose) isomerase, glucose oxidase, and/or peroxide-degrading enzyme disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix). Non conservative substitutions are amino acid substitutions that are not conservative substitutions.

In certain embodiments, a disclosed glucose(xylose) isomerase, glucose oxidase, and/or peroxide-degrading enzyme (or a pharmaceutical composition, e.g., a solid pharmaceutical composition, comprising the same) has a shelf-life (e.g., retains at least 30%, at least 40%, at least 50%, at least 60%, at least 70%, at least 80%, at least 85%, at least 90%, at least 95%, at least 96%, at least 97%, at least 98%, at least 99%, or 100% of its biological activity) at room temperature of at least 3 months, at least 6 months, at least 9 months, at least 12 months, at least 15 months, at least 18 months, at least 21 months, at least 24 months, at least 36 months, at least 48 months, at least 60 months, at least 72 months, at least 84 months, at least 96 months, at least 108 months, or at least 120 months. In certain embodiments, a disclosed glucose(xylose) isomerase, glucose oxidase, and/or peroxide-degrading enzyme (or a pharmaceutical composition, e.g., a solid pharmaceutical composition, comprising the same) may have a shelf-life at room temperature of from about 3 to about 120 months, from about 3 to about 96 months, from about 3 to about 72 months, from about 3 to about 48 months, from about 3 to about 24 months, from about 3 to about 21 months, from about 3 to about 18 months, from about 3 to about 15 months, from about 3 to about 12 months, from about 3 to about 9 months, from about 3 to about 6 months, from about 6 to about 120 months, from about 6 to about 96 months, from about 6 to about 72 months, from about 6 to about 48 months, from about 6 to about 24 months, from about 6 to about 21 months, from about 6 to about 18 months, from about 6 to about 15 months, from about 6 to about 12 months, from about 6 to about 9 months, from about 9 to about 120 months, from about 9 to about 96 months, from about 9 to about 72 months, from about 9 to about 48 months, from about 9 to about 24 months, from about 9 to about 21 months, from about 9 to about 18 months, from about 9 to about 15 months, from about 9 to about 12 months, from about 12 to about 120 months, from about 12 to about 96 months, from about 12 to about 72 months, from about 12 to about 48 months, from about 12 to about 24 months, from about 12 to about 21 months, from about 12 to about 18 months, from about 12 to about 15 months, from about 15 to about 120 months, from about 15 to about 96 months, from about 15 to about 72 months, from about 15 to about 48 months, from about 15 to about 24 months, from about 15 to about 21 months, from about 15 to about 18 months, from about 18 to about 120 months, from about 18 to about 96 months, from about 18 to about 72 months, from about 18 to about 48 months, from about 18 to about 24 months, from about 18 to about 21 months, from about 21 to about 120 months, from about 21 to about 96 months, from about 21 to about 72 months, from about 21 to about 48 months, from about 21 to about 24 months, from about 24 to about 120 months, from about 24 to about 96 months, from about 24 to about 72 months, from about 24 to about 48 months, from about 48 to about 120 months, from about 48 to about 96 months, from about 48 to about 72 months, from about 72 to about 120 months, from about 72 to about 96 months, or from about 96 to about 120 months.

Glucose(xylose) isomerase, glucose oxidase, and/or peroxide-degrading enzyme stability, shelf-life, or half-life may be measured by any method known in the art, including enzymatic activity or SDS-PAGE analysis following incubation of an enzyme at selected conditions (e.g., temperature, pH, and/or humidity conditions) for a selected time period. It is understood that glucose(xylose) isomerase, glucose oxidase, and/or peroxide-degrading enzyme stability, shelf-life, or half-life will depend upon the experimental conditions in which it is measured.

In certain embodiments, the glucose(xylose) isomerase, glucose oxidase, and/or peroxide-degrading enzyme has at least 80%, 85%, 90%, 95%, 96%, 97%, 98%, or 99% sequence identity to a glucose(xylose) isomerase, glucose oxidase, and/or peroxide-degrading enzyme disclosed herein.

For example, in certain embodiments, the glucose(xylose) isomerase enzyme has at least 80%, 85%, 90%, 95%, 96%, 97%, 98%, or 99% sequence identity to SEQ ID NO: 5. In certain embodiments, the glucose oxidase enzyme has at least 80%, 85%, 90%, 95%, 96%, 97%, 98%, or 99% sequence identity to SEQ ID NO: 1 or SEQ ID NO: 2. In certain embodiments, the peroxide-degrading enzyme has at least 80%, 85%, 90%, 95%, 96%, 97%, 98%, or 99% sequence identity to SEQ ID NO: 3 or SEQ ID NO: 4. Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases, see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

It is contemplated that a disclosed glucose(xylose) isomerase, glucose oxidase, and/or peroxide-degrading enzyme may be modified, engineered or chemically conjugated. For example, it is contemplated that a disclosed glucose(xylose) isomerase, glucose oxidase, and/or peroxide-degrading enzyme can be conjugated to an effector agent using standard in vitro conjugation chemistries. If the effector agent is a polypeptide, the glucose(xylose) isomerase, glucose oxidase, and/or peroxide-degrading enzyme can be chemically conjugated to the effector or joined to the effector as a fusion protein. Construction of fusion proteins is within ordinary skill in the art.

In certain embodiments, depending upon a particular mode of administration or site of activity, a disclosed glucose(xylose) isomerase, glucose oxidase, and/or peroxide-degrading enzyme can be modified with a moiety that improves its stabilization and/or retention in circulation, e.g., in blood, serum, or other tissues. For example, a disclosed glucose(xylose) isomerase, glucose oxidase, and/or peroxide-degrading enzyme may be conjugated to a polymer, e.g., a substantially non-antigenic polymer, such as a polyalkylene oxide or a polyethylene oxide. In certain embodiments, a disclosed glucose(xylose) isomerase, glucose oxidase, and/or peroxide-degrading enzyme is conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof. Additional useful polymers include polyoxyalkylenes such as polyoxyethylene, polyoxypropylene, and block copolymers of polyoxyethylene and polyoxypropylene, polymethacrylates, carbomers, and branched or unbranched polysaccharides.

### II. Enzyme Production

Methods for producing glucose(xylose) isomerase, glucose oxidase, and/or peroxide-degrading enzymes of the invention are known in the art. For example, DNA molecules encoding a glucose(xylose) isomerase, glucose oxidase, and/or peroxide-degrading enzyme can be chemically synthesized using the sequence information provided herein. Synthetic DNA molecules can be ligated to other appropriate nucleotide sequences, including, e.g., expression control sequences, to produce conventional gene expression constructs encoding the desired glucose(xylose) isomerase, glucose oxidase, and/or peroxide-degrading enzyme.

Nucleic acids encoding desired glucose(xylose) isomerase, glucose oxidase, and/or peroxide-degrading enzymes can be incorporated (ligated) into expression vectors, which can be introduced into host cells through conventional transfection or transformation techniques. Transformed host cells can be grown under conditions that permit the host cells to express the genes that encode the glucose(xylose) isomerase, glucose oxidase, and/or peroxide-degrading enzyme.

In certain embodiments, nucleic acids encoding recombinant glucose(xylose) isomerase, glucose oxidase, and/or peroxide-degrading enzymes of the invention may be codon optimized for expression in a heterologous cell, e.g., a yeast cell (e.g. a Pichia cell) or an E. coli cell, using methods known in the art.

Specific expression and purification conditions will vary depending upon the expression system employed. For example, if a gene is to be expressed in E. coli, it can be cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

A glucose(xylose) isomerase, glucose oxidase, and/or peroxide-degrading enzyme can be produced by growing (culturing) a host cell transfected with an expression vector encoding such glucose(xylose) isomerase, glucose oxidase, and/or peroxide-degrading enzyme, under conditions that permit expression of the glucose(xylose) isomerase, glucose oxidase, and/or peroxide-degrading enzyme. Following expression, the glucose(xylose) isomerase, glucose oxidase, and/or peroxide-degrading enzyme can be harvested and purified or isolated using techniques known in the art, e.g., affinity tags such as glutathione-S-transferase (GST) and histidine tags.

In certain embodiments, a glucose(xylose) isomerase, glucose oxidase, and/or peroxide-degrading enzyme is dried, e.g., spray-dried. Pharmaceutical proteins may be dried in many ways, e.g., by removal of water, organic solvent or liquid polymer by means including drying with N₂, air or inert gases, vacuum oven drying, lyophilization, washing with a volatile organic solvent followed by evaporation of the solvent, evaporation in a fume hood, tray drying, fluid bed drying, spray drying, vacuum drying, or roller drying.

Spray drying glucose(xylose) isomerase, glucose oxidase, and/or peroxide-degrading enzymes allows water to be separated from the glucose(xylose) isomerase, glucose oxidase, and/or peroxide-degrading enzyme preparation, allowing for continuous production of dry solids in powder, granulate, or agglomerate form from liquid feedstocks such as emulsions and pumpable suspensions. Spray drying involves the atomization of a liquid feedstock comprising glucose(xylose) isomerase, glucose oxidase, and/or peroxide-degrading enzyme into a spray of droplets and contacting the droplets with hot air or gas in a drying chamber. The atomization process may be conducted using a two-fluid atomizer that mixes the liquid feedstock with a drying gas such as compressed air or nitrogen. Operating conditions and dryer design are selected according to the drying characteristics of the glucose(xylose) isomerase, glucose oxidase, and/or peroxide-degrading enzyme and the desired powder qualities. Exemplary methods for spray drying enzymes are described in United States Patent Application Publication No. 2015/0353913. The glucose(xylose) isomerase, glucose oxidase, and peroxide-degrading enzyme may be spray dried separately or combined and spray dried together.

In certain embodiments, a glucose(xylose) isomerase, glucose oxidase, and/or peroxide-degrading enzyme is immobilized. For example, a glucose(xylose) isomerase, glucose oxidase, and/or peroxide-degrading enzyme may be immobilized on IRA-904 resin, a calcium alginate bead, Ludox HS-30, or a silica bead.

### III. Pharmaceutical Compositions

For therapeutic use, a glucose(xylose) isomerase, glucose oxidase, and/or peroxide-degrading enzyme described herein preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing a naturally occurring or recombinant glucose(xylose) isomerase, glucose oxidase, and/or peroxide-degrading enzyme disclosed herein can be presented in a dosage unit form and can be prepared by any suitable method. A pharmaceutical composition should be formulated to be compatible with its intended route of administration. The pharmaceutical compositions may be in a variety of forms. These include, for example, liquid, semi-solid and solid dosage forms, such as liquid solutions, dispersions or suspensions, tablets, pills, powders, liposomes and suppositories. The preferred form will depend upon the intended mode of administration and therapeutic application.

Although the compositions preferably are formulated for administration enterally (for example, orally), such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and infrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Depending upon the mode of administration, for example, by parenteral administration, it may be desirable to produce a pharmaceutical formulation that is sterile. Sterilization can be accomplished by any suitable method, e.g., filtration through sterile filtration membranes. Where the composition is lyophilized, filter sterilization can be conducted prior to or following lyophilization and reconstitution.

In certain embodiments, a disclosed composition comprises a polyionic reagent which may, e.g., coat the glucose(xylose) isomerase, glucose oxidase, and/or peroxide-degrading enzyme (e.g., the composition comprises a polyionic coating). Exemplary polyionic reagents include PSS (poly(Sodium 4-styrenesulfonate), PAA (poly Acrylic acid sodium salt), PMG (poly(methylene-co-guanidine) hydrochloride), DS (dextran sulfate), PMA (poly(methyl acrylate)), or PVS (polyvinylsiloxane).

In certain embodiments, a disclosed composition and/or dosage comprises: (i) about 750 to about 250,000, about 750 to about 200,000, about 750 to about 150,000, about 750 to about 100,000, 750 to about 75,000, about 750 to about 60,000, about 750 to about 45,000, about 750 to about 30,000, about 750 to about 15,000, about 750 to about 10,000, about 750 to about 7,500, about 750 to about 5,000, about 750 to about 2,500, about 750 to about 1,000, about 1,000 to about 250,000, about 1,000 to about 200,000, about 1,000 to about 150,000, about 1,000 to about 100,000, 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 250,000, about 2,500 to about 200,000, about 2,500 to about 150,000, about 2,500 to about 100,000, 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 250,000, about 5,000 to about 200,000, about 5,000 to about 150,000, about 5,000 to about 100,000, 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 250,000, about 7,500 to about 200,000, about 7,500 to about 150,000, about 7,500 to about 100,000, 7,500 to about 75,000, about 7,500 to about 60,000, about 7,500 to about 45,000, about 7,500 to about 30,000, about 7,500 to about 15,000, about 7,500 to about 10,000, about 10,000 to about 250,000, about 10,000 to about 200,000, about 10,000 to about 150,000, about 10,000 to about 100,000, 10,000 to about 75,000, about 10,000 to about 60,000, about 10,000 to about 45,000, about 10,000 to about 30,000, about 10,000 to about 15,000, about 15,000 to about 250,000, about 15,000 to about 200,000, about 15,000 to about 150,000, about 15,000 to about 100,000, 15,000 to about 75,000, about 15,000 to about 60,000, about 15,000 to about 45,000, about 15,000 to about 30,000, about 30,000 to about 250,000, about 30,000 to about 200,000, about 30,000 to about 150,000, about 30,000 to about 100,000, about 30,000 to about 75,000, about 30,000 to about 60,000, about 30,000 to about 45,000, about 45,000 to about 250,000, about 45,000 to about 200,000, about 45,000 to about 150,000, about 45,000 to about 100,000, about 45,000 to about 75,000, about 45,000 to about 60,000, about 60,000 to about 250,000, about 60,000 to about 200,000, about 60,000 to about 150,000, about 60,000 to about 100,000, about 60,000 to about 75,000, about 75,000 to about 250,000, about 75,000 to about 200,000, about 75,000 to about 150,000, about 75,000 to about 100,000, about 100,000 to about 250,000, about 100,000 to about 200,000, about 100,000 to about 150,000, about 150,000 to about 250,000, about 150,000 to about 200,000, about 200,000 to about 250,000 international units (I.U.) of glucose(xylose) isomerase enzyme; and/or (ii) about 750 to about 250,000, about 750 to about 200,000, about 750 to about 150,000, about 750 to about 100,000, 750 to about 75,000, about 750 to about 60,000, about 750 to about 45,000, about 750 to about 30,000, about 750 to about 15,000, about 750 to about 10,000, about 750 to about 7,500, about 750 to about 5,000, about 750 to about 2,500, about 750 to about 1,000, about 1,000 to about 250,000, about 1,000 to about 200,000, about 1,000 to about 150,000, about 1,000 to about 100,000, 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 250,000, about 2,500 to about 200,000, about 2,500 to about 150,000, about 2,500 to about 100,000, 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 250,000, about 5,000 to about 200,000, about 5,000 to about 150,000, about 5,000 to about 100,000, 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 250,000, about 7,500 to about 200,000, about 7,500 to about 150,000, about 7,500 to about 100,000, 7,500 to about 75,000, about 7,500 to about 60,000, about 7,500 to about 45,000, about 7,500 to about 30,000, about 7,500 to about 15,000, about 7,500 to about 10,000, about 10,000 to about 250,000, about 10,000 to about 200,000, about 10,000 to about 150,000, about 10,000 to about 100,000, 10,000 to about 75,000, about 10,000 to about 60,000, about 10,000 to about 45,000, about 10,000 to about 30,000, about 10,000 to about 15,000, about 15,000 to about 250,000, about 15,000 to about 200,000, about 15,000 to about 150,000, about 15,000 to about 100,000, 15,000 to about 75,000, about 15,000 to about 60,000, about 15,000 to about 45,000, about 15,000 to about 30,000, about 30,000 to about 250,000, about 30,000 to about 200,000, about 30,000 to about 150,000, about 30,000 to about 100,000, about 30,000 to about 75,000, about 30,000 to about 60,000, about 30,000 to about 45,000, about 45,000 to about 250,000, about 45,000 to about 200,000, about 45,000 to about 150,000, about 45,000 to about 100,000, about 45,000 to about 75,000, about 45,000 to about 60,000, about 60,000 to about 250,000, about 60,000 to about 200,000, about 60,000 to about 150,000, about 60,000 to about 100,000, about 60,000 to about 75,000, about 75,000 to about 250,000, about 75,000 to about 200,000, about 75,000 to about 150,000, about 75,000 to about 100,000, about 100,000 to about 250,000, about 100,000 to about 200,000, about 100,000 to about 150,000, about 150,000 to about 250,000, about 150,000 to about 200,000, about 200,000 to about 250,000 international units (I.U.) of glucose oxidase enzyme; and/or (iii) about 750 to about 250,000, about 750 to about 200,000, about 750 to about 150,000, about 750 to about 100,000, 750 to about 75,000, about 750 to about 60,000, about 750 to about 45,000, about 750 to about 30,000, about 750 to about 15,000, about 750 to about 10,000, about 750 to about 7,500, about 750 to about 5,000, about 750 to about 2,500, about 750 to about 1,000, about 1,000 to about 250,000, about 1,000 to about 200,000, about 1,000 to about 150,000, about 1,000 to about 100,000, 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 250,000, about 2,500 to about 200,000, about 2,500 to about 150,000, about 2,500 to about 100,000, 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 250,000, about 5,000 to about 200,000, about 5,000 to about 150,000, about 5,000 to about 100,000, 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 250,000, about 7,500 to about 200,000, about 7,500 to about 150,000, about 7,500 to about 100,000, 7,500 to about 75,000, about 7,500 to about 60,000, about 7,500 to about 45,000, about 7,500 to about 30,000, about 7,500 to about 15,000, about 7,500 to about 10,000, about 10,000 to about 250,000, about 10,000 to about 200,000, about 10,000 to about 150,000, about 10,000 to about 100,000, 10,000 to about 75,000, about 10,000 to about 60,000, about 10,000 to about 45,000, about 10,000 to about 30,000, about 10,000 to about 15,000, about 15,000 to about 250,000, about 15,000 to about 200,000, about 15,000 to about 150,000, about 15,000 to about 100,000, 15,000 to about 75,000, about 15,000 to about 60,000, about 15,000 to about 45,000, about 15,000 to about 30,000, about 30,000 to about 250,000, about 30,000 to about 200,000, about 30,000 to about 150,000, about 30,000 to about 100,000, about 30,000 to about 75,000, about 30,000 to about 60,000, about 30,000 to about 45,000, about 45,000 to about 250,000, about 45,000 to about 200,000, about 45,000 to about 150,000, about 45,000 to about 100,000, about 45,000 to about 75,000, about 45,000 to about 60,000, about 60,000 to about 250,000, about 60,000 to about 200,000, about 60,000 to about 150,000, about 60,000 to about 100,000, about 60,000 to about 75,000, about 75,000 to about 250,000, about 75,000 to about 200,000, about 75,000 to about 150,000, about 75,000 to about 100,000, about 100,000 to about 250,000, about 100,000 to about 200,000, about 100,000 to about 150,000, about 150,000 to about 250,000, about 150,000 to about 200,000, about 200,000 to about 250,000 international units (I.U.) of peroxide-degrading enzyme.

Depending upon the circumstances, the ratio of the number of international units of glucose(xylose) isomerase to the number of international units of the glucose oxidase enzyme is in the range of 0.1 to 10, for example, 0.1 to 9, 0.1 to 8, 0.1 to 7, 0.1 to 6, 0.1 to 5, 0.1 to 4, 0.1 to 3, 0.1 to 1 or 0.1 to 1, 0.5 to 10, 0.5 to 9, 0.5 to 8, 0.5 to 7, 0.5 to 6, 0.5 to 5, 0.5 to 4, 0.5 to 3 or 0.5 to 2, 0.5 to 1, 1 to 10, 1 to 9, 1 to 8, 1 to 7, 1 to 6, 1 to 5, 1 to 4, 1 to 3 or 1 to 2.

Depending upon the circumstances, the ratio of the number of international units of glucose oxidase to the number of international units of the peroxidase-degrading enzyme is in the range of 0.1 to 10, for example, 0.1 to 9, 0.1 to 8, 0.1 to 7, 0.1 to 6, 0.1 to 5, 0.1 to 4, 0.1 to 3, 0.1 to 1 or 0.1 to 1, 0.5 to 10, 0.5 to 9, 0.5 to 8, 0.5 to 7, 0.5 to 6, 0.5 to 5, 0.5 to 4, 0.5 to 3 or 0.5 to 2, 0.5 to 1, 1 to 10, 1 to 9, 1 to 8, 1 to 7, 1 to 6, 1 to 5, 1 to 4, 1 to 3 or 1 to 2.

Depending upon the circumstances, the amount of glucose(xylose) isomerase, glucose oxidase, and/or peroxidase degrading enzyme administered to a subject in a particular dosing can range from about 50 to about 20,000, about 50 to about 15,000, about 50 to about 10,000, about 50 to about 7,500, about 50 to about 5,000, about 50 to about 4,000, about 50 to about 3000, about 50 to about 2000, about 50 to about 1,000, about 50 to about 750, about 50 to about 500, or about 50 to about 150 international units/kg, for example, about 20,000, about 15,000, about 10,000, about 5,000, about 4,000, about 3,000, 2,000, 1,000 international units/kg.

For a 180 kg human, it is contemplated that about 500,000 to about 4,000,000, 500,000 to about 3,000,000, about 500,000 to about 2,000,000, 500,000 to about 1,000,000, 1,000,000 to about 4,000,000 1,000,000 to about 3,000,000, or 1,000,000 to about 2,000,000 international units of glucose(xylose) isomerase, glucose oxidase, and/or peroxidase degrading enzyme may be administered in a single dose.

In certain embodiments, a disclosed glucose(xylose) isomerase, glucose oxidase, and/or peroxide-degrading enzyme or composition is administered to a subject together with a meal or snack. In certain embodiments, a disclosed glucose(xylose) isomerase, glucose oxidase, and/or peroxide-degrading enzyme or composition is administered to a subject together with each meal or snack that the subject eats. In certain embodiments, a disclosed glucose(xylose) isomerase, glucose oxidase, and/or peroxide-degrading enzyme or composition is administered to a subject once every 7 days, once every 6 days, once every 5 days, once every 4 days, once every 3 days, once every 2 days, once every day, 2 times every day, 3 times every day, 4 times every day, 5 times every day, 6 times every, or more than 6 times every day.

Depending upon the circumstances, the composition can be formulated as a powder, granulate, pellet, micropellet, or a minitablet. The composition can be encapsulated in a capsule, e.g., a hydroxypropyl methylcellulose (HPMC) capsule, soft gelatin capsule, or a hard gelatin capsule. Alternatively, the composition can be formulated as a tablet dosage form. The composition may also be formulated as a liquid oral dosage form, for example, an elixir, syrup, or drop.

### IV. Therapeutic Uses

The methods and compositions disclosed herein can be used to treat diseases or disorders. For example, the invention provides a method of treating fructose intolerance (e.g., dietary fructose intolerance or hereditary fructose intolerance), irritable bowel syndrome (IBS), FODMAP (fermentable oligosaccharides, disaccharides, monosaccharides, and polyols) malabsorption and/or intolerance, obesity, metabolic syndrome, diabetes, hyperglycemia, or hyperinsulinemia. The method comprises administering to the subject an effective amount of (i) a glucose(xylose) isomerase enzyme, (ii) a glucose oxidase enzyme, and/or (iii) a peroxide-degrading enzyme. For example, the method may comprise administering a disclosed pharmaceutical composition comprising (i) a spray dried or lyophilized glucose(xylose) isomerase, (ii) a spray-dried or lyophilized glucose oxidase enzyme, and/or (iii) a spray-dried or lyophilized peroxide-degrading enzyme.

The term “effective amount” as used herein refers to the amount of an active agent (e.g., a disclosed glucose(xylose) isomerase, glucose oxidase, and/or peroxide-degrading enzyme) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

The invention also provides a method of reducing a level of fructose in a subject, for example, a subject with fructose intolerance (e.g., dietary fructose intolerance or hereditary fructose intolerance), irritable bowel syndrome (IBS), FODMAP (fermentable oligosaccharides, disaccharides, monosaccharides, and polyols) malabsorption and intolerance, obesity, metabolic syndrome, diabetes, hyperglycemia, or hyperinsulinemia. The method comprises administering to the subject an effective amount of (i) a glucose(xylose) isomerase, a (ii) glucose oxidase enzyme, and/or (iii) a peroxide-degrading enzyme. For example, the method may comprise administering a disclosed pharmaceutical composition comprising (i) a spray dried or lyophilized glucose(xylose) isomerase, (ii) a spray-dried or lyophilized glucose oxidase, and/or (ii) a spray-dried or lyophilized peroxide-degrading enzyme. A level of fructose in a subject may refer to a level of fructose in a body fluid (e.g., blood, plasma, serum, or urine), tissue, organ, cell, and/or gastrointestinal tract in the subject, or a sample of any of the foregoing.

Fructose levels, e.g., blood fructose levels, may be measured by any method known in the art. Exemplary fructose measuring kits are available from Sigma Aldrich (FA20-1KT) and Biovision (K439-100).

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This Example describes a study testing the effect of glucose(xylose) isomerase on the conversion of fructose in the absence and in the presence of glucose oxidase.

The materials used in this and the following Examples were as follows: fructose (Sigma, Cat. No. F2543-100G), glucose (Sigma Cat. No. G8270-100G), phosphate buffer saline (GIBCO Life Technologies Cat. No. 70011-044), sodium acetate (Emplura MERC Cat. No. 1.93644.0521G), glucose assay kit (Lyphochek glucose or Lyphozyme from Agappe Diagnostics Ltd. or from Beacon Diagnostics Pvt. Ltd., respectively), fructose assay kit (Sigma Cat. No. FA20-1KT), glucose isomerase (Sigma Cat. No. G4166-50G), glucose oxidase (Sigma Cat. No. G7141-10KU), and catalase (Cat. No. C3515-25MG). Except where indicated otherwise, all reagents and kits were used in accordance with the manufacturer's instructions.

Fructose (1% solution) was incubated with glucose(xylose) isomerase and/or glucose oxidase, as depicted in Table 1, and the conversion of fructose to glucose by glucose(xylose) isomerase and degradation of glucose to gluconic acid and hydrogen peroxide by glucose oxidase was monitored. Assays were carried in both acetate buffer, pH 5.0 and phosphate buffered saline (PBS), pH 7.4 in order to mimic the pH conditions of the intestine.

TABLE 1
Experimental Procedure

| | | Enzyme | |
|---|---|---|---|
| | | Glucose(xylose) | Enzyme |
| | | Isomerase | Glucose Oxidase |
| | Substrate | (10 μL, Final | (10 μL, Final |
| Test | Fructose | Concentration 0.3 | Concentration 0.4 |
| Sample | (1 mL, 1%) | Units/mL) | Units/mL) |
| 1 | PBS, pH 7.4 | + | − |
| 2 | Acetate, pH 5.0 | + | − |
| 3 | PBS, pH 7.4 | − | + |
| 4 | Acetate, pH 5.0 | − | + |
| 5 | PBS, pH 7.4 | + | + |
| 6 | Acetate, pH 5.0 | + | + |

Test samples were prepared in 2 mL Eppendorf tubes and incubated at 37° C. with constant stirring. Aliquots were withdrawn at 2 and 4 hours and filtered through a centrifugal filter (0.2 μm) to separate glucose(xylose) isomerase enzyme (as it is immobilized) to prevent further conversion of fructose to glucose. The samples were then tested for glucose and fructose concentration using a corresponding assay kit.

The results are presented for glucose in Table 2 and for fructose in Table 3. The data indicates that glucose(xylose) isomerase alone converts fructose to glucose to equilibrium as there was no further decrease in fructose concentration after 2 hours and no further increase in glucose concentration after 2 hours. However, in the presence of glucose oxidase there was further reduction of glucose due to degradation of glucose by glucose oxidase. The results demonstrate that a mixture of glucose(xylose) isomerase and glucose oxidase converted or degraded more than 70 to 80% of fructose in solution within a 3 to 4 hour time period at pH 5.0 and 7.4.

TABLE 2
Experimental Results - Glucose Concentration

Glucose Concentration
(mg/100 mL)

| Test Sample | 2 hours | 4 hours |
|---|---|---|
| 1 | 481 | 515 |
| 2 | 549 | 571 |
| 3 | 0.79 | ND |
| 4 | 1.17 | ND |
| 5 | 421 | 16.7 |
| 6 | 375 | 15.6 |

TABLE 3
Experimental Results - Fructose Concentration

| | Fructose Concentration | | |
|---|---|---|---|
| | (mg/100 mL) | | |
| Test Sample | 2 hours | 4 hours | 6 hours |
| 1 | 703 | 672 | 680 |
| 2 | 714 | 726 | 734 |
| 3 | 1033 | ND | ND |
| 4 | 1067 | ND | ND |
| 5 | 544 | 270 | 131 |
| 6 | 452 | 222 | 126 |

### Example 2

This Example describes a study testing the effect of glucose(xylose) isomerase on the conversion of fructose in the absence and in the presence of glucose oxidase and catalase.

Fructose (1% solution) was incubated with glucose(xylose) isomerase, glucose oxidase, and/or catalase, as depicted in Table 4, and the conversion of fructose to glucose by glucose(xylose) isomerase, and degradation of glucose to gluconic acid and hydrogen peroxide by glucose oxidase was monitored. Assays were carried in both acetate buffer, pH 5.0 and phosphate buffered saline (PBS), pH 7.4 in order to mimic the pH conditions of the intestine.

TABLE 4
Experimental Procedure

| | | Enzyme | | |
|---|---|---|---|---|
| | | Glucose(xylose) | Enzyme | Enzyme |
| | | Isomerase | Glucose Oxidase | Catalase |
| | Substrate | (10 μL, Final | (10 μL, Final | (25 μL, Final |
| Test | Fructose | Concentration 3 | Concentration 4 | Concentration 10 |
| Sample | (1 mL, 1%) | Units/mL) | Units/mL) | Units/mL) |
| 1 | PBS, pH 7.4 | + | − | − |
| 2 | Acetate, pH 5.0 | + | − | − |
| 3 | PBS, pH 7.4 | − | + | − |
| 4 | Acetate, pH 5.0 | − | + | − |
| 5 | PBS, pH 7.4 | + | + | − |
| 6 | Acetate, pH 5.0 | + | + | − |
| 7 | PBS, pH 7.4 | + | + | + |
| 8 | Acetate, pH 5.0 | + | + | + |

Test samples were prepared in 2 mL Eppendorf tubes and incubated at 37° C. with constant stirring. Aliquots were withdrawn at 1, 2, 3 and 4 hours and filtered through a centrifugal filter (0.2 pm) to separate glucose(xylose) isomerase enzyme (as it is immobilized) to prevent further conversion of fructose to glucose. The samples were then tested for glucose and fructose concentration using a corresponding assay kit.

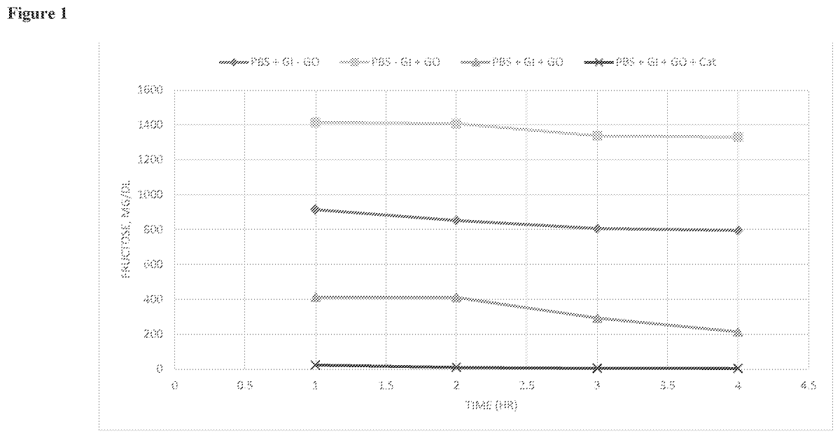

The results are presented for glucose in Table 5 and for fructose in Table 6. Results at pH 7.4 are also shown in FIG. 1. As depicted, the presence of catalase improved the degradation of fructose. Incubation with glucose(xylose) isomerase and glucose oxidase for 1 hour resulted in 42% (pH 7.4) or 34% (pH 5.0) of glucose remaining. However, incubation with glucose(xylose) isomerase, glucose oxidase, and catalase for 1 hour resulted in 2% (pH 7.4) or 0.2% (pH 5.0) of glucose remaining. Unexpectedly, better results were seen at pH 5.0 relative to pH 7.4. It is hypothesized this may be due to the pH profile of glucose oxidase and catalase, but not glucose(xylose) isomerase (which is expected to perform better at pH 7.4). These results suggest that, in vivo, a combination of glucose(xylose) isomerase, glucose oxidase, and catalase may be active in the stomach.

TABLE 5
Experimental Results - Glucose Concentration

| | |
|---|---|
| Test | Glucose Concentration (mg/100 mL) |

| Sample | 1 hour | 2 hours | 3 hours | 4 hours |
|---|---|---|---|---|
| 1 | 576 | 527 | 547 | 541 |
| 2 | 538 | 562 | 549 | 549 |
| 3 | 1.55 | 3.74 | 1.96 | 1.79 |
| 4 | 1.68 | 1.83 | 2.04 | 1.55 |
| 5 | 416 | 395 | 331 | 318 |
| 6 | 343 | 274 | 268 | 250 |
| 7 | 21.43 | 3.95 | 2.16 | 1.93 |
| 8 | 2.83 | 1.77 | 1.53 | 1.77 |

TABLE 6
Experimental Results - Glucose Concentration

| | |
|---|---|
| Test | Fructose Concentration (mg/100 mL) |

| Sample | 1 hour | 2 hours | 3 hours | 4 hours |
|---|---|---|---|---|
| 1 | 913.77 | 851.61 | 805.60 | 794.73 |
| 2 | ND | 840.19 | ND | ND |
| 3 | 1413.81 | 1407.26 | 1336.71 | 1329.44 |
| 4 | ND | 1324.97 | ND | ND |
| 5 | 412.90 | 410.15 | 292.00 | 213.39 |
| 6 | 436.26 | 355.07 | 136.52 | 19.42 |
| 7 | 23.43 | 9.79 | 4.83 | 3.93 |
| 8 | 5.95 | 4.16 | 3.77 | 3.53 |

SEQUENCE LISTING

SEQ ID NO: 1 (Aspergillus niger glucose oxidase; Uniprot P13006):
MQTLLVSSLVVSLAAALPHYIRSNGIEASLLTDPKDVSGRTVDYIIAGGGLTGLTTAARLTENP
NISVLVIESGSYESDRGPIIEDLNAYGDIFGSSVDHAYETVELATNNQTALIRSGNGLGGSTLV
NGGTWTRPHKAQVDSWETVFGNEGWNWDNVAAYSLQAERARAPNAKQIAAGHYFNASCHGVNGT
VHAGPRDTGDDYSPIVKALMSAVEDRGVPTKKDFGCGDPHGVSMFPNTLHEDQVRSDAAREWLL
PNYQRPNLQVLTGQYVGKVLLSQNGTTPRAVGVEFGTHKGNTHNVYAKHEVLLAAGSAVSPTIL
EYSGIGMKSILEPLGIDTVVDLPVGLNLQDQTTATVRSRITSAGAGQGQAAWFATFNETFGDYS
EKAHELLNTKLEQWAEEAVARGGFHNTTALLIQYENYRDWIVNHNVAYSELFLDTAGVASFDVW
DLLPFTRGYVHILDKDPYLHHFAYDPQYFLNELDLLGQAAATQLARNISNSGAMQTYFAGETIP
GDNLAYDADLSAWTEYIPYHFRPNYHGVGTCSMMPKEMGGVVDNAARVYGVQGLRVIDGSIPPT
QMSSHVMTVFYAMALKISDAILEDYASMQ
SEQ ID NO: 2 (Aspergillus niger glucose oxidase; Uniprot P13006, w/o signal
sequence):
SNGIEASLLTDPKDVSGRTVDYIIAGGGLTGLTTAARLTENPNISVLVIESGSYESDRGPIIED
LNAYGDIFGSSVDHAYETVELATNNQTALIRSGNGLGGSTLVNGGTWTRPHKAQVDSWETVFGN
EGWNWDNVAAYSLQAERARAPNAKQIAAGHYFNASCHGVNGTVHAGPRDTGDDYSPIVKALMSA
VEDRGVPTKKDFGCGDPHGVSMFPNTLHEDQVRSDAAREWLLPNYQRPNLQVLTGQYVGKVLLS
QNGTTPRAVGVEFGTHKGNTHNVYAKHEVLLAAGSAVSPTILEYSGIGMKSILEPLGIDTVVDL
PVGLNLQDQTTATVRSRITSAGAGQGQAAWFATFNETFGDYSEKAHELLNTKLEQWAEEAVARG
GFHNTTALLIQYENYRDWIVNHNVAYSELFLDTAGVASFDVWDLLPFTRGYVHILDKDPYLHHF
AYDPQYFLNELDLLGQAAATQLARNISNSGAMQTYFAGETIPGDNLAYDADLSAWTEYIPYHFR
PNYHGVGTCSMMPKEMGGVVDNAARVYGVQGLRVIDGSIPPTQMSSHVMTVFYAMALKISDAIL
EDYASMQ
SEQ ID NO: 3 (Aspergillus niger catalase; Uniprot A0A254TZH3):
MRHFWLLPAVAGIAGAQCPYLSGEMSFTQEQDNAGDTIEVTEQPIDNTLYVNDTGSYMTTDFGT
PISDQTSLKAGPRGPTLLEDFIFRQKLQRFDHERVPERVVHARGAGAYGTFKSYADWSNVTAAD
FLSANDKETPMFCRFSTVVGFRGSVDTARDVHGHACRFYTDEGNYDIVGINFAPFFIQDAIQFP
DLVHAIKPMPNNEIPQAATAHTSAWDFFSQQSTALHSALWLMSGNGIPRSFRHMNGYGVHSFRF
VAANGTSKVVRYRWKSQQGVASLVWDEAQAAAGKNSDYHRQDLYNAIANGHYPKYELQAQIMDE
ADMLRFGFDLLDPTKLVPEEVVPYTPLGMMELNANPTNYFAEVEQAGFQPGHVVPGIDFTDDPL
LQGRLFSYLDTQLTRHGGPNFEQIPVNRPRKPVHNNNRDGFGQQQIPTNNWAYTPNSMSNGYPM
QANQTQGHGFFTAPYRYASGHLVRQTSPTFNDHWSQPAMFWNSLIPAEQQMVVNAIVFENSKVN
SPHVRKNVVNQLNMVNNNLAVRVARGLGLDEPSPNPTYYTSNKTSNVGTFGKPLLSIEGLQVGF
LASNSHPESIKQGQAMAAQFSAAGVDLNIVTEAYADGVNTTYALSDAIDFDALIIADGVQSLFA
SPALANQMNSTATSTLYPPARPFQILVDSFRYGKPVAAVGSGSVALKNAGIDSSRSGVYTGSSE
TTEKIAKEVLEGLYTFRFVDRFALDE
SEQ ID NO: 4 (Aspergillus niger catalase; Uniprot A0A254TZH3; w/o signal
sequence):
QCPYLSGEMSFTQEQDNAGDTIEVTEQPIDNTLYVNDTGSYMTTDFGTPISDQTSLKAGPRGPT
LLEDFIFRQKLQRFDHERVPERVVHARGAGAYGTFKSYADWSNVTAADFLSANDKETPMFCRFS
TVVGFRGSVDTARDVHGHACRFYTDEGNYDIVGINFAPFFIQDAIQFPDLVHAIKPMPNNEIPQ
AATAHTSAWDFFSQQSTALHSALWLMSGNGIPRSFRHMNGYGVHSFRFVAANGTSKVVRYRWKS
QQGVASLVWDEAQAAAGKNSDYHRQDLYNAIANGHYPKYELQAQIMDEADMLRFGFDLLDPTKL
VPEEVVPYTPLGMMELNANPTNYFAEVEQAGFQPGHVVPGIDFTDDPLLQGRLFSYLDTQLTRH
GGPNFEQIPVNRPRKPVHNNNRDGFGQQQIPTNNWAYTPNSMSNGYPMQANQTQGHGFFTAPYR
YASGHLVRQTSPTFNDHWSQPAMFWNSLIPAEQQMVVNAIVFENSKVNSPHVRKNVVNQLNMVN
NNLAVRVARGLGLDEPSPNPTYYTSNKTSNVGTFGKPLLSIEGLQVGFLASNSHPESIKQGQAM
AAQFSAAGVDLNIVTEAYADGVNTTYALSDAIDEDALIIADGVQSLFASPALANQMNSTATSTL
YPPARPFQILVDSFRYGKPVAAVGSGSVALKNAGIDSSRSGVYTGSSETTEKIAKEVLEGLYTF
RFVDRFALDE
SEQ ID NO: 5 (Streptomyces murinus glucose(xylose) isomerase; Uniprot P37031):
MSFQPTPEDRFTFGLWTVGWQGRDPFGDATRPALDPVETVQRLAELGAYGVTFHDDDLIPFGSS
DTERESHIKRFRQALDATGMTVPMATTNLFTHPVFKDGGFTANDRDVRRYALRKTIGNIDLAAE
LGAKTYVAWGGREGAESGGAKDVRDALDRMKEAFDLLGEYVTAQGYDLRFAIEPKPNEPRGDIL
LPTVGHALAFIERLERPELYGVNPEVGHEQMAGLNFPHGIAQALWAGKLFHIDLNGQSGIKYDQ
DLRFGAGDLRAAFWLVDLLETAGYEGPRHFDFKPPRTEDFDGVWASAAGCMRNYLILKDRAAAF
RADPEVQEALRAARLDQLAQPTAADGLDALLADRAAFEDFDVDAAAARGMAFEHLDQLAMDHLL
GARG
SEQ ID NO: 6:
VX₁WX₂GREGX₃E
X₁ is any amino acid, X₂ is G or P, X₃ is Y, S, T, A, or E
SEQ ID NO: 7:
X₁EPKPX₂X₃P
X₁ is L, I, V, or M, X₂ is any amino acid, X₃ is E or Q

What is claimed is:

1. A pharmaceutical composition comprising a glucose(xylose) isomerase enzyme, a glucose oxidase enzyme, a peroxide-degrading enzyme, and a pharmaceutically acceptable excipient.

2. The pharmaceutical composition of claim 1, wherein the glucose(xylose) isomerase enzyme is spray-dried or lyophilized.

3. The pharmaceutical composition of claim 1 or 2, wherein the glucose(xylose) isomerase enzyme is a microbial glucose(xylose) isomerase enzyme, or a functional fragment or variant thereof.

4. The pharmaceutical composition of any one of claims 1-3, wherein the glucose(xylose) isomerase enzyme is derived from Streptomyces murinus.

5. The pharmaceutical composition of claim 4, wherein the glucose(xylose) isomerase enzyme comprises SEQ ID NO: 5, or a functional fragment or variant thereof.

6. The pharmaceutical composition of any one of claims 1-5, wherein the composition comprises from about 1,000 to about 250,000 international units (I.U.) of the glucose(xylose) isomerase enzyme.

7. The pharmaceutical composition of any one of claims 1-6, wherein the glucose oxidase enzyme is spray-dried or lyophilized.

8. The pharmaceutical composition of any one of claims 1-7, wherein the glucose oxidase enzyme is a microbial glucose oxidase enzyme, or a functional fragment or variant thereof.

9. The pharmaceutical composition of any one of claims 1-8, wherein the glucose oxidase enzyme is derived from Aspergillus niger.

10. The pharmaceutical composition of claim 9, wherein the glucose oxidase enzyme comprises SEQ ID NO: 2, or a functional fragment or variant thereof.

11. The pharmaceutical composition of any one of claims 1-10, wherein the composition comprises from about 1,000 to about 250,000 international units (I.U.) of the glucose oxidase enzyme.

12. The pharmaceutical composition of any one of claims 1-11, wherein the peroxide-degrading enzyme is spray-dried or lyophilized.

13. The pharmaceutical composition of any one of claims 1-2, wherein the peroxide-degrading enzyme is a catalase enzyme or a peroxidase enzyme.

14. The pharmaceutical composition of claim 13, wherein the peroxide-degrading enzyme is a catalase enzyme.

15. The pharmaceutical composition of claim 14, wherein the catalase enzyme is a microbial catalase enzyme, or a functional fragment or variant thereof.

16. The pharmaceutical composition of claim 14 or 15, wherein the catalase enzyme is derived from Aspergillus niger.

17. The pharmaceutical composition of claim 16, wherein the catalase enzyme comprises SEQ ID NO: 4, or a functional fragment or variant thereof.

18. The pharmaceutical composition of any one of claims 1-12, wherein the composition comprises from about 1,000 to about 2,500,000 international units (I.U.) of the peroxide-degrading enzyme.

19. The pharmaceutical composition of any one of claims 1-18, wherein the ratio of international units of the glucose(xylose) isomerase to international units of the glucose oxidase enzyme is in the range from 0.1 to 10.

20. The pharmaceutical composition of any one of claims 1-19, wherein the ratio of international units of glucose oxidase to international units of the peroxide-degrading enzyme is in the range from 0.1 to 10.

21. The pharmaceutical composition of any one of claims 1-20, wherein the composition is formulated as an oral dosage form (e.g., a solid or liquid oral dosage form).

22. The pharmaceutical composition of claim 21, wherein the composition is a formulated as a powder, satchel, granulate, pellet, micropellet, tablet, minitablet, elixir, syrup, or drop.

23. The pharmaceutical composition of claim 21 or 22, wherein the composition is formulated as a powder, satchel, or tablet.

24. The pharmaceutical composition of any one of claims 1-23, wherein the composition has a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

25. A method of treating fructose intolerance in a subject in need thereof, the method comprising orally administering to the subject the pharmaceutical composition of any one of claims 1-24.

26. The method of claim 25, wherein the fructose intolerance is hereditary fructose intolerance.

27. The method of claim 25, wherein the fructose intolerance is dietary fructose intolerance.

28. A method of reducing a fructose level in a subject, the method comprising orally administering to the subject the pharmaceutical composition of any one of claims 1-24.

29. The method of claim 28, wherein the method reduces the level of fructose in the blood of the subject and/or in the gastrointestinal tract of the subject.

30. The method of claim 28 or 29, wherein the method reduces the level of fructose in a sample from the subject.

31. The method of claim 30, wherein the sample is a body fluid sample.

32. The method of claim 31, wherein the body fluid sample is blood, serum or plasma.

33. The method of any one of claims 28-32, wherein the method reduces the level of fructose in the blood of the subject from 0 to 240 minutes or from 0 to 360 minutes following a meal or snack, as determined by an Area Under Curve (AUC) analysis, by at least about 10%, 20%, 30%, 40%, 50%, 60%, 70%, 80%, or 90%.

34. The method of any one of claims 28-33, wherein the method reduces fructose Cmax in the blood of the subject following a meal or snack by at least about 10%, 20%, 30%, 40%, 50%, 60%, 70%, 80%, or 90%.

35. The method of any one of claims 28-34, wherein the method reduces fructose Tmax in the blood of the subject following a meal or snack by at least about 10%, 20%, 30%, 40%, 50%, 60%, 70%, 80%, or 90%.

36. The method of any one of claims 28-35, wherein, when administered together with a food, the method reduces the caloric intake of the food by at least about 10%, 20%, 30%, 40%, 50%, 60%, 70%, 80%, or 90%.

37. The method of any one of claims 28-36, wherein the subject has dietary fructose intolerance, hereditary fructose intolerance, irritable bowel syndrome (IBS), FODMAP (fermentable oligosaccharides, disaccharides, monosaccharides, and polyols) intolerance or malabsorption, obesity, metabolic syndrome, diabetes, hyperglycemia, or hyperinsulinemia.

38. The method of any one of claims 25-37, wherein the pharmaceutical composition is administered to the subject 1, 2, 3, 4, or more than 4 times per day.

39. The method of any one of claims 25-38, wherein the pharmaceutical composition is administered to the subject together with a meal or snack.

40. The method of any one of claims 25-39, wherein the pharmaceutical composition is administered to the subject at each meal or snack.

41. The method of any one of claims 25-40, wherein the subject as a human adult or a human pediatric subject.


SEQUENCE LISTING (7 sequences)

SEQ ID NO: 1 - protein, 605 aa, synthetic construct
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 2 - protein, 583 aa, synthetic construct
SNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 3 - protein, 730 aa, synthetic construct
MRHFWLLPAVAGIAGAQCPYLSGEMSFTQEQDNAGDTIEVTEQPIDNTLYVNDTGSYMTTDFGTPISDQTSLKAGPRGPTLLEDFIFRQKLQRFDHERVPERVVHARGAGAYGTFKSYADWSNVTAADFLSANDKETPMFCRFSTVVGFRGSVDTARDVHGHACRFYTDEGNYDIVGINFAPFFIQDAIQFPDLVHAIKPMPNNEIPQAATAHTSAWDFFSQQSTALHSALWLMSGNGIPRSFRHMNGYGVHSFRFVAANGTSKVVRYRWKSQQGVASLVWDEAQAAAGKNSDYHRQDLYNAIANGHYPKYELQAQIMDEADMLRFGFDLLDPTKLVPEEVVPYTPLGMMELNANPTNYFAEVEQAGFQPGHVVPGIDFTDDPLLQGRLFSYLDTQLTRHGGPNFEQIPVNRPRKPVHNNNRDGFGQQQIPTNNWAYTPNSMSNGYPMQANQTQGHGFFTAPYRYASGHLVRQTSPTFNDHWSQPAMFWNSLIPAEQQMVVNAIVFENSKVNSPHVRKNVVNQLNMVNNNLAVRVARGLGLDEPSPNPTYYTSNKTSNVGTFGKPLLSIEGLQVGFLASNSHPESIKQGQAMAAQFSAAGVDLNIVTEAYADGVNTTYALSDAIDFDALIIADGVQSLFASPALANQMNSTATSTLYPPARPFQILVDSFRYGKPVAAVGSGSVALKNAGIDSSRSGVYTGSSETTEKIAKEVLEGLYTFRFVDRFALDE

SEQ ID NO: 4 - protein, 714 aa, synthetic construct
QCPYLSGEMSFTQEQDNAGDTIEVTEQPIDNTLYVNDTGSYMTTDFGTPISDQTSLKAGPRGPTLLEDFIFRQKLQRFDHERVPERVVHARGAGAYGTFKSYADWSNVTAADFLSANDKETPMFCRFSTVVGFRGSVDTARDVHGHACRFYTDEGNYDIVGINFAPFFIQDAIQFPDLVHAIKPMPNNEIPQAATAHTSAWDFFSQQSTALHSALWLMSGNGIPRSFRHMNGYGVHSFRFVAANGTSKVVRYRWKSQQGVASLVWDEAQAAAGKNSDYHRQDLYNAIANGHYPKYELQAQIMDEADMLRFGFDLLDPTKLVPEEVVPYTPLGMMELNANPTNYFAEVEQAGFQPGHVVPGIDFTDDPLLQGRLFSYLDTQLTRHGGPNFEQIPVNRPRKPVHNNNRDGFGQQQIPTNNWAYTPNSMSNGYPMQANQTQGHGFFTAPYRYASGHLVRQTSPTFNDHWSQPAMFWNSLIPAEQQMVVNAIVFENSKVNSPHVRKNVVNQLNMVNNNLAVRVARGLGLDEPSPNPTYYTSNKTSNVGTFGKPLLSIEGLQVGFLASNSHPESIKQGQAMAAQFSAAGVDLNIVTEAYADGVNTTYALSDAIDFDALIIADGVQSLFASPALANQMNSTATSTLYPPARPFQILVDSFRYGKPVAAVGSGSVALKNAGIDSSRSGVYTGSSETTEKIAKEVLEGLYTFRFVDRFALDE

SEQ ID NO: 5 - protein, 388 aa, synthetic construct
MSFQPTPEDRFTFGLWTVGWQGRDPFGDATRPALDPVETVQRLAELGAYGVTFHDDDLIPFGSSDTERESHIKRFRQALDATGMTVPMATTNLFTHPVFKDGGFTANDRDVRRYALRKTIGNIDLAAELGAKTYVAWGGREGAESGGAKDVRDALDRMKEAFDLLGEYVTAQGYDLRFAIEPKPNEPRGDILLPTVGHALAFIERLERPELYGVNPEVGHEQMAGLNFPHGIAQALWAGKLFHIDLNGQSGIKYDQDLRFGAGDLRAAFWLVDLLETAGYEGPRHFDFKPPRTEDFDGVWASAAGCMRNYLILKDRAAAFRADPEVQEALRAARLDQLAQPTAADGLDALLADRAAFEDFDVDAAAARGMAFEHLDQLAMDHLLGARG

SEQ ID NO: 6 - protein, 10 aa, synthetic construct
  VARIANT 2: note = X can be any amino acid
  VARIANT 4: note = G or P
  VARIANT 9: note = Y,S,T, A, or E
VXWXGREGXE

SEQ ID NO: 7 - protein, 8 aa, synthetic construct
  VARIANT 6: note = X can be any amino acid
  VARIANT 7: note = E or Q
  VARIANT 1: note = L, I, V or M
XEPKPXXP